(19) United States
(12) Patent Application Publication
(10) Pub. No.: US 2024/0257350 A1
(43) Pub. Date: Aug. 1, 2024

(54) PROVIDING A RESULT DATA SET

(71) Applicant: Siemens Healthineers AG, Forchheim (DE)

(72) Inventors: Markus Kowarschik, Erlangen (DE); Stephanie Hench, Trailsdorf (DE); Angelika Hench, Hallerndorf-Trailsdorf (DE); Marcus Pfister, Bubenreuth (DE)

(21) Appl. No.: 18/426,070

(22) Filed: Jan. 29, 2024

(30) Foreign Application Priority Data
Jan. 31, 2023 (DE) 10 2023 200 770.3

(51) Int. Cl. G06T 7/00; G16H 30/20; G16H 30/40

(52) U.S. Cl. CPC G06T 7/0016; G16H 30/20; G16H 30/40; G06T 2207/10116; G06T 2207/20081; G06T 2207/30004

(57) ABSTRACT

A method for providing a result data set includes: capturing a first medical image data set that maps an object under examination within a first temporal phase; capturing a second medical image data set that maps a flow of contrast agent in the object under examination within a second temporal phase in a time-resolved manner; identifying multiple partial image data sets in the second image data set, wherein the partial image data sets in each case map one of multiple physiological subphases within the second temporal phase; and providing the result data set including multiple subtraction image data sets, wherein each subtraction image data set of the multiple subtraction image data sets is determined based on the first medical image data set and a respective partial image data set of the multiple partial image data sets in the second medical image data set.

The present patent document claims the benefit of German Patent Application No. 10 2023 200 770.3, filed Jan. 31, 2023, which is hereby incorporated by reference in its entirety.

### TECHNICAL FIELD

The present disclosure relates to a method for providing a result data set, a method for providing a comparison data set, a method for providing a trained function, a provision unit, a medical imaging device, and a computer program product.

### BACKGROUND

X-ray-based imaging methods are frequently employed for capturing changes over time to a field of examination of an object under examination. The change over time to be captured may include a propagation motion and/or flow motion of a contrast agent, in particular of a contrast agent flow and/or a contrast agent bolus, in a hollow organ, (e.g., a vascular section), of the object under examination.

The X-ray-based imaging methods may include digital subtraction angiography (DSA), wherein at least two X-ray images taken in a temporal sequence, which at least partially map the common field of examination, are subtracted from one another. Furthermore, in DSA, a distinction is frequently made between a mask phase for acquiring at least one mask image and a filling phase for acquiring at least one filling image. In this case, the mask image may map the field of examination in an uncontrasted manner, in particular without contrast agent. Further, the filling image may map the field of examination in a contrasted manner, in particular while the contrast agent is disposed in the field of examination. As a result of the DSA, a difference image is frequently provided by subtraction of mask image and filling image. Consequently, the components in the difference image, which in particular are unchanging over time and which are irrelevant to a treatment and/or diagnostic investigation and/or cause interference, may be reduced and/or removed.

DSA represents a central imaging technique for supporting endovascular interventions. Such interventions may include procedures in which at least partially occluded vessels are opened, in particular a recanalization and/or mechanical thrombectomy for ischemic stroke, and/or procedures in which vessels are occluded, in particular an embolization and/or chemoembolization in the case of a hepatocellular carcinoma (HCC).

The success of a therapy frequently has to be evaluated by a medical operator, (e.g., a physician and/or interventionalist and/or radiologist), by considering DSA series. The disadvantage of this is that contrasted vessels, (e.g., arterial and/or venous vessels), may overlay a contrasted tissue of the object under examination, making exclusive consideration of the tissue more difficult. This may apply to various body regions of the object under examination, for example, the brain.

Thanks to a manual adjustment of parameters, by which the DSA series are displayed, in particular thanks to a parameterization of gray-scale windows (center/width), the dedicated consideration of the tissue may be improved. However, manual adjustment is time-consuming and prone to error. What is known as a TICI score (Thrombolysis in Cerebral Infarction) is frequently of importance in the treatment of strokes. A reperfusion of brain tissue involved may be evaluated here on the basis of DSA images, which requires external windowing of the images. However, a contrasted parenchyma of the object under examination is frequently overlaid with contrasted vessels of the object under examination.

### SUMMARY AND DESCRIPTION

It is hence the object of the present disclosure to enable a specific mapping of a contrasted field of examination of an object under examination. The scope of the present disclosure is defined solely by the appended claims and is not affected to any degree by the statements within this summary. The present embodiments may obviate one or more of the drawbacks or limitations in the related art.

The achievement of the object both in respect of methods and devices for providing a result data set and in respect of methods and devices for providing a trained function is described below. Features, advantages, and alternative forms of embodiment of data structures and/or functions in the case of methods and devices for providing a result data set may here be transferred to analogous data structures and/or functions in the case of methods and devices for providing a trained function. Analogous data structures may here in particular be characterized by the use of the prefix “training.” Furthermore, the trained functions used in methods and devices for providing a result data set may be adjusted and/or provided by methods and devices for providing a trained function.

A first aspect relates to an, in particular computer-implemented, method for providing a result data set. In this case, a first medical image data set is captured, which maps an object under examination within a first temporal phase. Further, a second medical image data set is captured, which maps a flow of contrast agent in the object under examination within a second temporal phase in a time-resolved manner. Furthermore, multiple partial image data sets are identified in the second image data set. The partial image data sets in each case form one of multiple physiological subphases within the second temporal phase. After this, the result data set is provided. The result data set includes multiple subtraction image data sets, which are determined on the basis of the first image data set and in each case one of the partial image data sets.

The above-described acts of the proposed method may be computer-implemented in part or in full. Furthermore, the above-described acts of the proposed method may be carried out at least in part, in particular in full, consecutively, or at least in part simultaneously.

The capture of the first and/or second medical image data set may in each case include a receipt and/or acquisition of the respective image data set. The receipt of the first and/or second medical image data set may include a capture and/or readout of a computer-readable data store and/or a receipt from a data memory unit, for example, a database. Further, the first and/or second medical image data set may be provided by a provision unit of a medical imaging device. Alternatively, or additionally, the first and/or second medical image data set may be acquired by a medical imaging device. In particular, the first and second medical image data set may be acquired by the same medical imaging device or different medical imaging devices.

The medical imaging device for acquiring the first and/or second image data set may include a medical X-ray device, (e.g., a medical C-arm X-ray device), a cone-beam computed tomography facility (cone-beam CT, CBCT), a computed tomography facility (CT facility), a magnetic resonance tomography facility (MRT facility), a positron emission tomography facility (PET facility), an ultrasound device, or a combination thereof.

The object under examination may include a male or female human, an animal patient, or an examination phantom, in particular, a vascular phantom. Further, the object under examination may have a field of examination. The field of examination may include a spatial section, in particular a volume, of the object under examination, which has a hollow organ and/or tissue. The hollow organ may include a vascular section, in particular an artery and/or vein. Further, the tissue may include a parenchyma.

The first image data set may advantageously include a two-dimensionally (2D) and/or three-dimensionally (3D) spatially resolved mapping of the object under examination, in particular of the field of examination. The first image data set may map the object under examination within the first temporal phase, in particular a mask phase. For this, the first image data set may be acquired within a predefined first period. The first image data set may be reconstructed from multiple first individual images, in particular multiple first projection mappings, which each have a mapping of at least one section of the object under examination.

The second image data set may advantageously include a 2D and/or 3D spatially resolved mapping of the object under examination, in particular of the field of examination. Further, the second image data set maps the flow of contrast agent, in particular a flow motion and/or propagation motion of a contrast agent, in the object under examination, in particular in the hollow organ and/or tissue of the object under examination, in a time-resolved manner. The second image data set may further be reconstructed from multiple second individual images, in particular multiple second projection mappings, which each have a mapping of at least one section of the object under examination.

The first and second image data set may in each case include multiple image points, in particular pixels or voxels, with image values, for example attenuation values and/or intensity values, which map the object under examination.

The second image data set may map the object under examination within a second temporal phase, in particular a filling phase. For this, the second image data set may be acquired within a predefined second period. In this case, the second temporal phase may be downstream of the first temporal phase. The contrast agent, (e.g., an X-ray-opaque contrast agent), may advantageously be arranged in the object under examination, in particular the hollow organ and/or tissue only within the second temporal phase. Consequently, the object under examination may have a contrasted hollow organ and/or tissue within the second temporal phase.

Multiple partial image data sets are identified in the second image data set. The partial image data sets may have all features and properties that have been described in respect of the second image data set. Each of the partial image data sets may advantageously in each case map one of multiple physiological subphases within the second temporal phase. In particular, the partial image data sets may each, in particular at least in part or in full, map different physiological subphases within the second temporal phase.

The identification of the partial image data sets in the second image data set may advantageously take place automatically, for example, by applying a trained function and/or on the basis of the respective acquisition times and/or on the basis of distinguishing the respectively mapped flow of contrast agent. The partial image data sets may advantageously in each case have at least one of multiple mappings of a temporal sequence of mappings of the object under examination, which the second image data set includes. The identification of the partial image data sets may include a selection and/or annotation and/or provision of the partial image data sets in the second image data set.

The provision of the result data set may include storage on a computer-readable storage medium and/or display on a display unit and/or transfer to a provision unit. In particular, a graphical display of the result data set may be displayed by the display unit.

Advantageously, the provision of the result data set may include a provision of multiple subtraction image data sets. The subtraction image data sets may be determined on the basis of the first image data set and in each case one of the partial image data sets. In particular, a subtraction image data set may in each case be determined for each of the partial image data sets.

For example, the subtraction image data sets may in each case be determined as a difference, in particular image point by image point and/or area by area, between the first image data set and in each case one of the partial image data sets. Alternatively, the subtraction image data sets may in each case be determined as a difference, in particular image point by image point and/or area by area, between the first image data set and a processing result of processing in each case of one of the partial image data sets, for example, a maximum opacity image.

Using the proposed method, a specific mapping of a contrasted field of examination of the object under examination, for example a contrasted hollow organ and/or tissue, may advantageously be enabled.

In a further advantageous form of embodiment of the proposed method, the subtraction image data sets may be determined as a difference between the first image data set and in each case one of the partial image data sets.

Advantageously, the subtraction image data sets may be determined by subtraction, in particular pixel by pixel or voxel by voxel, of the first image data set from in each case one of the partial image data sets. In particular, a subtraction image data set may in each case be determined for each of the partial image data sets. In this case, image values, (e.g., attenuation values and/or intensity values), of corresponding image points, in particular pixels or voxels, of the first image data set may be subtracted from image values, (e.g., attenuation values and/or intensity values), of the respective partial image data set.

Using the proposed form of embodiment, parts of the object under examination, which are uncontrasted in the first temporal phase, may advantageously be removed from the subtraction image data sets. Furthermore, determining a subtraction image data set for the physiological subphases in each case enables a phase-specific consideration of the contrasted field of examination of the object under examination. In particular, interfering superimpositions of contrasted hollow organs and/or tissues of the object under examination from multiple physiological subphases may advantageously be mitigated or prevented.

In a further advantageous form of embodiment of the proposed method, the provision of the result data set may include a determination in each case of a maximum opacity image for the partial image data sets, which image point by image point has a maximum opacity within the respective physiological subphase along a temporal dimension. Further, the subtraction image data sets may be determined as a difference between the first image data set and in each case one of the maximum opacity images.

Advantageously, the maximum opacity images may include a determination, image point by image point, (e.g., pixel by pixel or voxel by voxel), of a maximum opacity, (e.g., X-ray attenuation), along a temporal dimension of the second image data set within the respective physiological subphase. If the second image data set includes multiple X-ray images, in particular multiple X-ray projection mappings, the maximum opacity images may be determined by determination, image point by image point, of a maximum X-ray attenuation along the associated beam over time. Advantageously, in each case, a maximum opacity image may be determined for each of the physiological subphases.

Advantageously, the subtraction image data sets may be determined by subtraction, in particular pixel by pixel or voxel by voxel, of the first image data set from in each case one of the maximum opacity images. In particular, in each case, a subtraction image data set may be determined for each of the maximum opacity images.

The proposed form of embodiment may enable improved highlighting of contrasted areas, in particular of the hollow organ and/or tissue, of the object under examination.

In a further advantageous form of embodiment of the proposed method, the physiological subphases may include an arterial phase and/or a parenchymal phase and/or a venous phase.

The object under examination may, in the physiological subphases, have an at least partially different contrast-filling of the hollow organ, (e.g., an artery and/or vein), and/or of the tissue. In this case, the contrast-filling designates an at least partial, in particular complete, filling of the hollow organ and/or tissue with the contrast agent. In the arterial phase, predominantly arterial vascular sections of the object under examination, (e.g., one or more arteries), may be contrasted. In the venous phase, predominantly venous vascular sections of the object under examination, (e.g., one or more veins), may be contrasted. In the parenchymal phase, a blush, (e.g., an acinarization), of the parenchyma may dominate in comparison with contrast-filling of the arterial and venous vascular sections. In particular, in the parenchymal phase, as few arterial and venous vascular sections as possible may be contrasted. In particular, the arterial, the parenchymal, and the venous phases may be mapped consecutively in time in the second image data set. Further, the mask phase mapped in the first image data set may lie temporally before the arterial phase.

The proposed form of embodiment may advantageously enable a phase-specific consideration of contrasted areas of the object under examination. In particular, a dedicated consideration, in particular substantially free from superimposed structures, of the contrasted parenchyma may be enabled.

In a further advantageous form of embodiment of the proposed method, the identification of the multiple partial image data sets may include applying a trained function to input data. In this case, the input data may be based on the second image data set. At least one parameter of the trained function may be adjusted on the basis of a comparison of training image data sets with comparison partial image data sets. Furthermore, the multiple partial image data sets may be provided as output data of the trained function.

The trained function may advantageously be trained using a machine-learning method. In particular, the trained function may be a neural network, in particular a convolutional neuronal network (CNN) or a network including a convolutional layer.

The trained function maps input data to output data. In this case, the output data may further depend on one or more parameters of the trained function. The one or the multiple parameters of the trained function may be determined and/or adjusted by training. The determination and/or adjustment of the one or multiple parameters of the trained function may be based on a pair including training input data and associated training output data, (e.g., comparison output data), wherein the trained function is applied to the training input data for the generation of training mapping data. In particular, the determination and/or adjustment may be based on a comparison of the training mapping data and the training output data, in particular comparison output data. A trainable function, (e.g., a function with one or more parameters that have not yet been adjusted), may be designated as a trained function.

Other terms for trained functions are trained mapping rule, mapping rule with trained parameters, function with trained parameters, and machine-learning algorithm. One example of a trained function is an artificial neural network, wherein edge weights of the artificial neural network correspond to the parameters of the trained function. Instead of the term “neural network,” the term “neural net” may also be used. In particular, a trained function may also be a deep neural network (deep artificial neural network). A further example of a trained function is a “support vector machine,” and furthermore other machine-learning algorithms may be employed as a trained function.

The trained function may be trained by backpropagation. Initially, training mapping data may be determined by applying the trained function to the training input data. After this, a deviation between the training mapping data and the training output data, in particular the comparison output data, may be determined by applying an error function to the training mapping data and the training output data, in particular the comparison output data. Further, at least one parameter, in particular a weighting of the trained function, may be iteratively adjusted. Consequently, the deviation between the training mapping data and the training output data, in particular the comparison output data, may be minimized during the training of the trained function.

Advantageously, the trained function, in particular the neural network, has an input layer and an output layer. In this case, the input layer may be configured to receive input data. Further, the output layer may be configured to provide mapping data, in particular the output data. In this case, the input layer and/or the output layer may each include multiple channels, in particular neurons.

The input data of the trained function may be based on the second image data set or may include the second image data set. Further, the trained function may provide the multiple partial image data sets as output data. Advantageously, at least one parameter of the trained function may be adjusted on the basis of a comparison of training image data sets with comparison partial image data sets. In particular, the trained function may be provided by a form of embodiment of the proposed method for providing a trained function, which is described below. The proposed form of embodiment may advantageously enable a compute-efficient identification of the partial image data sets.

In a further advantageous form of embodiment of the proposed method, the provision of the result data set may include a display of a graphical representation of the result data set by a display unit.

The display unit may include a monitor and/or projector and/or a display and/or data glasses, which are configured to display the graphical representation of the result data set. Advantageously, the provision of the result data set may include a display of a graphical representation of the subtraction image data sets. Provided that the result data set includes maximum opacity images, the provision of the result data set may include a display of a graphical representation of the maximum opacity images by the display unit.

Consequently, the result data set may advantageously be provided to a medical operator, (e.g., a physician), for visual consideration and diagnostic support.

In a further advantageous form of embodiment of the proposed method, the graphical representation of the result data set may include a color-coded and/or superimposed and/or coordinated and/or sequential representation of the subtraction image data sets.

Advantageously, the graphical representation of the result data set may include a representation of the subtraction image data sets that is color-coded in respect of the physiological subphases. Further, the graphical representation may include an at least partial, in particular full, superimposition of the subtraction image data sets. The superimposition of the subtraction image data sets may take place partially transparently or non-transparently. Furthermore, the graphical representation of the result data set may include a color-coded superimposition of the subtraction image data sets.

Alternatively, or additionally, the graphical representation of the result data set may include a coordinated representation of the subtraction image data sets, in particular an in particular tile-shaped arrangement, adjacent to one another and/or above one another, of the subtraction image data sets. Alternatively, or additionally, the provision of the result data set may include a sequential, in particular a temporally sequential, display of the graphical representations of the subtraction image data sets. In this case, the graphical representations of the subtraction image data sets may advantageously be displayed sequentially in accordance with the physiological subphases, in particular in accordance with a sequence of the physiological subphases.

Consequently, an improved distinguishability of the phase-specific subtraction image data sets for a cross-phase comparison, for example, by the medical personnel, may be enabled.

In a further advantageous form of embodiment of the proposed method, the provision of the result data set may include a registration of the partial image data set to be subtracted in each case and of the first image data set.

Advantageously, the partial image data set to be subtracted in each case and the first image data set may be registered with one another, in particular on the basis of common geometrical and/or anatomical features. The common geometrical features may include edges and/or contours and/or a marker structure and/or a contrast transition, which are mapped in the first image data set and the partial image data set. The common anatomical features may include a tissue border, an anatomical landmark, (e.g., an ostium), an implant, or a combination thereof, which are mapped in the first image data set and the partial image data set.

The registration of the first image data set and of the respective partial image data set may include applying a transformation, (e.g., a rigid or non-rigid transformation), such as a translation, a rotation, a scaling, a deformation, or a combination thereof, to the first image data set and/or the respective partial image data set, wherein a deviation between the common geometric and/or anatomical features is reduced, in particular minimized.

Consequently artifacts, (e.g., motion artifacts), in the subtraction image data sets may advantageously be minimized.

In a further advantageous form of embodiment of the proposed method, the partial image data sets may be identified on the basis of their respective acquisition times in the second image data set.

Advantageously, it is possible to identify, on the basis of the acquisition times of the second image data sets, in particular on the basis of temporal intervals between the acquisition times and/or a sequence of the acquisition times, which physiological subphase was mapped at the respective acquisition time. The times and/or intervals, in particular a start and/or end and/or a duration, of the respective subphase may be determined on the basis of pre-captured data of the object under examination and/or an operating parameter of a device for administration of the contrast agent, (e.g., an injection device), and/or on the basis of statistical data of a flow of contrast agent in objects under examination.

The proposed form of embodiment may advantageously enable a semiautomatic or automatic identification of the partial image data sets in the second image data set.

In a further advantageous form of embodiment of the proposed method, the partial image data sets may be identified on the basis of distinguishing the respectively mapped flow of contrast agent.

The identification of the partial image data sets on the basis of distinguishing the respectively mapped flow of contrast agent may be done manually, (e.g., by annotation), or automatically. For example, the partial image data sets, in particular the physiological subphases, may be identified on the basis of a fill-level of the hollow organ and/or tissue, and/or an arrangement of the contrast agent in the hollow organ and/or tissue. In particular, the partial image data sets, in particular the physiological subphases, may be identified on the basis of a substantially specific filling and/or coloration of a hollow organ and/or tissue of the object under examination with the contrast agent. The arterial phase may be identified on the basis of mapping a predominant contrast-filling of arterial vascular sections of the object under examination, in particular one or more arteries. The venous phase may be identified on the basis of mapping a predominant contrast-filling of venous vascular sections of the object under examination, in particular one or more veins. The parenchymal phase may be identified on the basis of mapping a dominant coloration of the parenchyma compared to a contrast-filling of the arterial and venous vascular sections.

The image-based identification of the partial image data sets in the second image data set may advantageously take place data-efficiently and/or computationally efficiently.

A second aspect relates to a method for providing a comparison data set. In this case, a first result data set is provided by performing a proposed method for providing a result data set at a first time. Further, a second result data set is provided by performing a proposed method for providing a result data set at a second time after the first time. In this case, a change in the object under examination has taken place between the first and the second time. After this, the comparison data set is provided, including in each case a difference between a subtraction image data set of the first and of the second result data set that map the same physiological subphase. The comparison data set may include a difference between a subtraction image data set of the first and of the second result data set that map the same physiological subphase. Alternatively, the comparison data set may include multiple differences between in each case a subtraction image data set of the first and of the second result data set, which in each case map the same physiological subphase.

Advantageously, the first and second image data sets of both the embodiments of the proposed method for providing a result data set may be captured with substantially the same or comparable acquisition parameters and/or injection parameters of the contrast agent. The change between the first and the second time may include an intervention and/or a surgical procedure on the object under examination, which is not part of the proposed method. For example, the first result data set may map the object under examination pre-interventionally, and the second result data set may map the object under examination post-interventionally. The comparison data set may in each case include a difference, in particular image point by image point, between respectively a subtraction image data set of the first and of the second result data set that map the same physiological subphase. For example, in each case a comparison data set may be provided for the arterial phase, the parenchymal phase and/or the venous phase.

The provision of the comparison data set may include storage on a computer-readable storage medium and/or display on a display unit and/or transfer to a provision unit. In particular, a graphical representation of the comparison data set may be displayed by the display unit.

The proposed method may advantageously enable a phase-specific comparison of the result data sets, for example, pre-interventionally, peri-interventionally, and/or post-interventionally. Consequently, an evaluation of the change, for example, of the success of an intervention, may advantageously be enabled.

A third aspect relates to a computer-implemented method for providing a trained function. In this case, a medical training image data set is captured, which maps a flow of contrast agent in an object under examination in a time-resolved manner. Further, multiple comparison partial image data sets are identified in the training image data set. The comparison partial image data sets in each case map one of multiple physiological subphases. Further, the comparison partial image data sets are identified in the training image data set on the basis of their respective acquisition times and/or on the basis of differences between the respectively mapped flow of contrast agent and/or by annotation. In a further act, multiple training image data sets are identified by applying the trained function to input data. In this case, the input data is based on the training image data set. The training image data sets are provided as output data of the trained function. Further, at least one parameter of the trained function is adjusted on the basis of a comparison between training image data sets and comparison partial image data sets. After this, the trained function is provided.

The capture of the medical training image data set may include a receipt and/or acquisition and/or simulation of the training image data set. The receipt of the medical training image data set may include a capture and/or readout of a computer-readable data store and/or a receipt from a data memory unit, for example, a database. Further, the medical training image data set may be provided by a provision unit of a medical imaging device. Alternatively, or additionally, the training image data set may be acquired by the medical imaging device. The medical training image data set may have all features and properties of the second medical image data set that have been described in respect of the proposed method for providing a result data set and vice versa. In particular, the medical training image data set may be a second medical image data set.

Alternatively, or additionally, the medical training image data set may be simulated, for example, the mapped flow of contrast agent in a model of a hollow organ and/or tissue of the object under examination, (e.g., a vascular section), may be simulated, for example, by computational fluid dynamics (CFD).

The comparison partial image data sets may have all features and properties of the partial image data sets that have been described in respect of the proposed method for providing a result data set and vice versa. The comparison partial image data sets may be identified on the basis of their respective acquisition times and/or on the basis of differences between the respectively mapped flow of contrast agent and/or by an annotation in the training image data set. The annotation may take place manually, for example, on the basis of a user input by an operator, by an input unit, or automatically.

Advantageously, the multiple training image data sets may be identified by applying the trained function to the input data. In this case, the input data of the trained function may be based on the training image data set, in particular may include the training image data set. Further, the trained function may provide the multiple training partial image data sets as output data.

By comparing the training partial image data sets with the comparison partial image data sets, in particular in each case for matching physiological subphases, the at least one parameter of the trained function may be adapted. The comparison may include determining a deviation between the training partial image data sets and the comparison partial image data sets, in particular between the image points of the training partial image data sets and the comparison partial image data sets. Alternatively, or additionally, the comparison may include determining a correlation, in particular a correlation value, between the training partial image data sets and the comparison partial image data sets. In this case, the at least one parameter of the trained function may advantageously be adjusted such that the deviation is minimized. The adjustment of the at least one parameter of the trained function may include optimizing, in particular minimizing, a cost value of a cost function, wherein the cost function characterizes, in particular quantifies, the deviation between the training partial image data sets and the comparison partial image data sets. In particular, adjusting the at least one parameter of the trained function may include a regression of the cost value of the trained function.

The provision of the trained function may include storage on a computer-readable storage medium and/or transfer to a provision unit.

Advantageously, the proposed method may be used to provide a trained function, which in one form of embodiment of the method may be used to provide a result data set.

A fourth aspect relates to a provision unit, which is configured to perform a proposed method for providing a result data set and/or for providing a comparison data set.

In this case, the provision unit may include a computing unit, a memory unit, and/or an interface. The provision unit may be configured to perform a proposed method for providing a result data set and/or for providing a comparison data set, in that the interface, the computing unit, and/or the memory unit are configured to perform the corresponding method acts.

In particular, the interface may be configured to capture the first and the second medical image data set and to provide the result data set. Further, the computing unit and/or the memory unit may be configured to identify the multiple partial image data sets. Furthermore, the interface may be configured to provide the comparison data set.

The advantages of the proposed provision unit may correspond to the advantages of the proposed method for providing a result data set and/or for providing a comparison data set. Features, advantages, or alternative forms of embodiment mentioned here may likewise also be transferred to the other claimed subject matters and vice versa.

The disclosure may further relate to a training unit configured to perform a proposed method for providing a trained function. In this case, the training unit may advantageously include a training interface, a training memory unit, and/or a training computing unit. The training unit may be configured to perform a method for providing a trained function, in that the training interface, the training memory unit, and/or the training computing unit are configured to perform the corresponding method acts. In particular, the training interface may be configured to capture the medical training image data set and to provide the trained function. Further, the training computing unit and/or the training memory unit may be configured to identify the multiple comparison data sets, identify the multiple training image data sets, and/or adjust the at least one parameter of the trained function.

The advantages of the proposed training unit may correspond to the advantages of the proposed method for providing a trained function. Features, advantages, or alternative forms of embodiment mentioned here may likewise also be transferred to the other claimed subject matters and vice versa.

A fifth aspect relates to a medical imaging device, including a proposed provision unit. In this case, the medical imaging device is configured to capture the first and second image data set.

The medical imaging device may be configured as a medical X-ray device, (e.g., a medical C-arm X-ray device), a cone-beam computed tomography facility (cone-beam CT, CBCT), a computed tomography facility (CT facility), a magnetic resonance tomography facility (MRT facility), a positron emission tomography facility (PET facility), an ultrasound device, or a combination thereof.

A sixth aspect relates to a computer program product with a computer program that may be loaded directly into a memory of a provision unit, with program sections in order to perform all acts of the method for providing a result data set and/or of the method for providing a comparison data set and/or the respective aspects thereof, if the program sections are executed by the provision unit; and/or which may be loaded directly into a training memory of a training unit, with program sections in order to execute all acts of a proposed method for providing a trained function and/or one aspect thereof, if the program sections are executed by the training unit.

The disclosure may further relate to a computer program or computer-readable storage medium, including a trained function that was provided by a proposed method or by one aspect thereof.

A largely software-based implementation has the advantage that provision units and/or training units already in use hitherto may easily be retrofitted by a software update, in order to work in the disclosed manner. Such a computer program product may include additional elements such as documentation and/or additional components, as well as hardware components such as hardware keys (e.g., dongles, etc.) for use of the software.

### BRIEF DESCRIPTION OF THE DRAWINGS

Exemplary embodiments of the disclosure are represented in the drawings and are described in greater detail below. The same reference characters are used in different figures for the same features. In the drawings:

FIG. 1 depicts a schematic representation of an embodiment of a method for providing a result data set.

FIG. 2 depicts a schematic representation of an additional embodiment of a method for providing a result data set.

FIG. 3 depicts an example of a schematic representation of a time axis with acquisition times of a first and second image data set.

FIG. 4 depicts an example of a schematic representation of maximum opacity images of different physiological subphases.

FIG. 5 depicts a schematic representation of an additional embodiment of a method for providing a result data set.

FIG. 6 depicts a schematic representation of an embodiment of a method for providing a comparison data set.

FIG. 7 depicts an example of a schematic representation of comparison data sets of different physiological subphases.

FIG. 8 depicts a schematic representation of an embodiment of a method for providing a trained function.

FIG. 9 depicts an example of a schematic representation of an artificial neural net.

FIG. 10 depicts an example of a schematic representation of a provision unit.

FIG. 11 depicts an example of a schematic representation of a training unit.

FIG. 12 depicts an example of a schematic representation of a medical imaging device.

### DETAILED DESCRIPTION

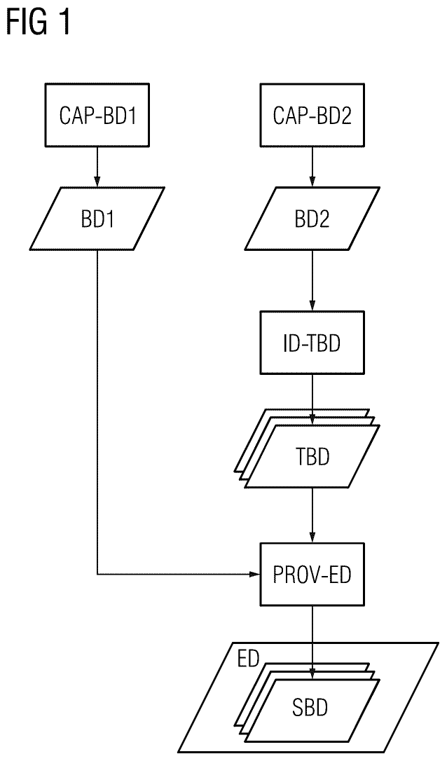

FIG. 1 schematically represents an advantageous form of embodiment of a proposed method for providing a result data set PROV-ED. In this case, a first medical image data set BD1 may be captured CAP-BD1, which maps an object under examination within a first temporal phase. Further, a second medical image data set BD2 may be captured CAP-BD2, which maps a flow of contrast agent in the object under examination within a second temporal phase in a time-resolved manner. After this, multiple partial image data sets TBD may be identified ID-TBD in the second image data set BD2. The partial image data sets TBD may be identified ID-TBD on the basis of their respective acquisition times and/or on the basis of differences between the respectively mapped flow of contrast agent in the second image data set BD2. The multiple partial image data sets TBD may in each case map one of multiple physiological subphases within the second temporal phase. The physiological subphases may include an arterial phase, a parenchymal phase, and/or a venous phase. After this, the result data set ED may be provided PROV-ED. In this case, the result data set ED may include multiple subtraction image data sets SBD, which are determined on the basis of the first image data set BD1 and in each case one of the partial image data sets TBD. In particular, the subtraction image data sets SBD may be determined as a difference between the first image data set BD1 and in each case one of the partial image data sets TBD.

Advantageously, the provision of the result data set PROV-ED may include a display of a graphical representation of the result data set ED by a display unit. In this case, the graphical representation of the result data set ED may include a color-coded and/or superimposed and/or coordinated and/or sequential representation of the subtraction image data sets SBD.

The provision of the result data set PROV-ED may further include a registration of the first image data set BD1 to be subtracted in each case and of the partial image data set TBD.

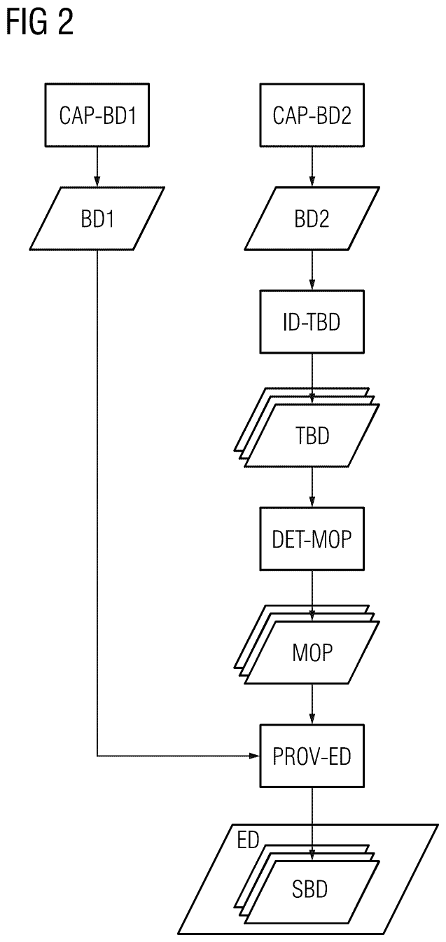

FIG. 2 shows a further advantageous form of embodiment of a proposed method for providing a result data set PROV-ED. In this case, the provision of the result data set PROV-ED may include a determination DET-MOP in each case of a maximum opacity image MOP for the partial image data sets TBD, which image point by image point has a maximum opacity within the respective physiological subphase along a temporal dimension. In this case, the subtraction image data sets SBD may be determined as the difference between the first image data set BD1 and in each case one of the maximum opacity images MOP.

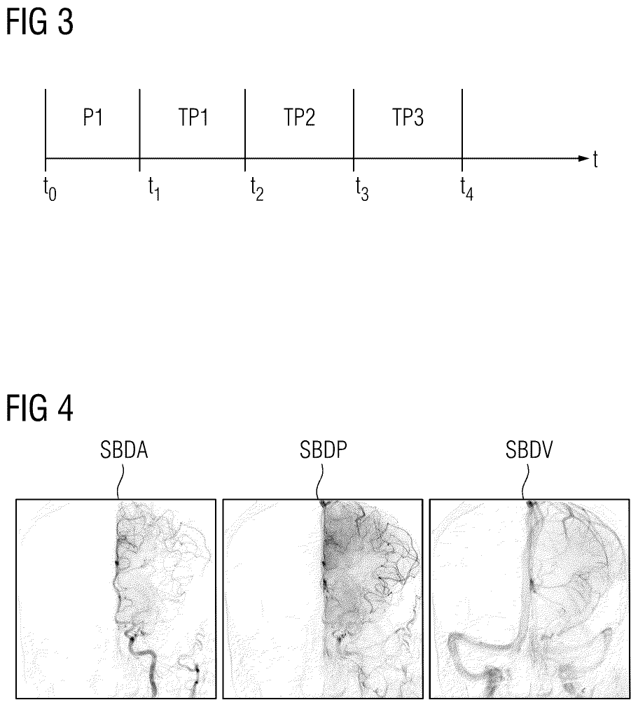

FIG. 3 shows a schematic representation of a time axis with acquisition times of a first and second image data set. The time t₀ may mark a commencement of the acquisition of the first image data set, in particular a triggering of X-ray radiation for acquiring the first image data set BD1. In particular, the time to may be known, for example, on the basis of acquisition parameters. The time t₁ may mark an inflow, (e.g., an arterial inflow), of contrast agent, in particular into the hollow organ, (e.g., an artery), of the object under examination, in particular a commencement of the arterial phase. The time t₁ may be automatically detected, for example, by automatic recognition of the inflowing contrast agent, in particular on the basis of a significant change in image values, in particular gray-scale values, in an image area of the second image data set BD2. Further, the time t₂ may mark a commencement of the parenchymal phase. Furthermore, the time t₃ may mark an outflow, (e.g., a venous outflow), of the contrast agent, in particular out of the hollow organ, (e.g., a vein), of the object under examination, in particular a commencement of the venous phase. The times t₂ and t₃ may be determined empirically in a first approximation, for example, on the basis of an image repetition rate (frames per second) and/or statistical measured values, in particular known from the literature. Supportively, or alternatively, image-based methods may also be used, for example, an evaluation of gradient-based metrics, since image gradients disappear due to the parenchyma blush. The time t₄ may mark an end of the venous phase.

Advantageously, the first image data set may be acquired within the first temporal phase, in particular between the times to and t₁. Further, the second temporal phase may be delimited by the times t₁ to t₄. In this case, the physiological subphases TP1, TP2, and TP3 may be identified within the second temporal phase. The first subphase TP1, in particular the arterial phase, may be delimited by the times t₁ to t₂. The second subphase TP2, in particular the parenchymal phase, may be delimited by the times t₂ to t₃. The third subphase TP3, in particular the venous phase, may be delimited by the times t₃ to t₄.

FIG. 4 shows a schematic representation of subtraction image data sets SBD of different physiological subphases. In this case, three subtraction image data sets SBDA, SBDP, and SBDV are represented as arranged next to one another by way of example. In FIG. 4, the subtraction image data set SBDA maps the arterial phase, the subtraction image data set SBDP maps the parenchymal phase, and the subtraction image data set SBDV maps the venous phase. In this case, the subtraction image data sets SBD have been determined as the difference between the first image data set BD1 and in each case a maximum opacity image MOP of the physiological subphase. In the subtraction image data set SBDA of the arterial phase, an occlusion of an arteria cerebri media (ACM) may be recognized and in the subtraction image data set SBDP of the parenchymal phase a resulting perfusion deficit in the media flow area may be recognized as a bright wedge in the contrasted hemisphere.

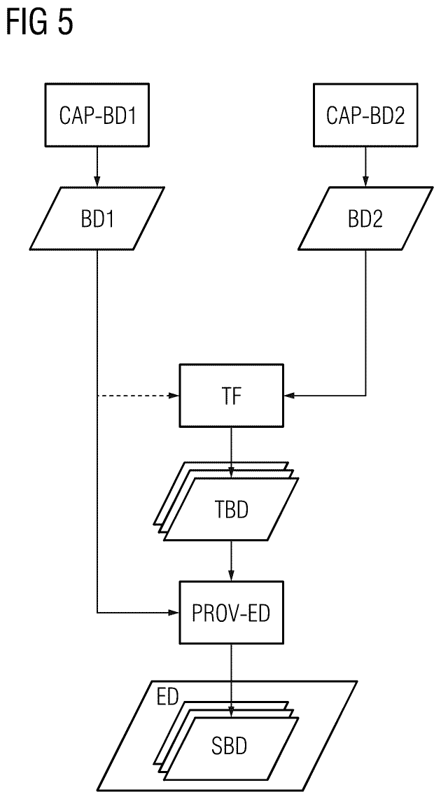

FIG. 5 shows a schematic representation of a further advantageous form of embodiment of a proposed method for providing a result data set PROV-ED. In this case, the identification ID-TBD of the multiple partial image data sets TBD may include applying a trained function TF to input data. In this case, the input data may be based on the second image data set BD2. Further, at least one parameter of the trained function TF may be adjusted on the basis of a comparison of training partial image data sets with comparison partial image data sets. Furthermore, the multiple partial image data sets TBD may be provided as output data of the trained function TF.

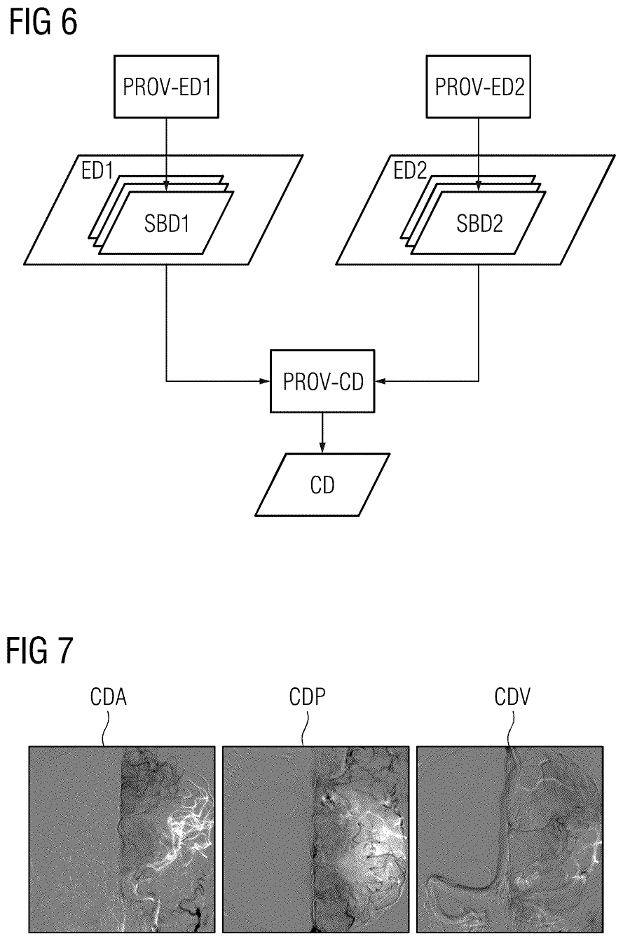

FIG. 6 shows a schematic representation of a proposed method for providing a comparison data set PROV-CD. In this case, a first result data set ED1 is provided PROV-ED1 by performing a method for providing a result data set at a first time. Further, a second result data set ED2 is provided PROV-ED2 by performing a method for providing a result data set at a second time after the first time. In this case, a change has taken place to the object under examination between the first and the second time. After this, the comparison data set CD including in each case a difference between a subtraction image data set SBD1 and SBD2 of the first and of the second result data set ED1 and ED2 is provided PROV-CD that map the same physiological subphase.

FIG. 7 shows a schematic representation of comparison data sets CD of different physiological subphases. In FIG. 7, the comparison data set CDA maps the arterial phase, the comparison data set CDP maps the parenchymal phase, and the comparison data set CDV maps the venous phase. The comparison data sets CDA, CBP, and CDV may be provided PROV-CD by subtraction of phase-specific subtraction image data sets SBD1 and SBD2 of the first and second result data set ED1 and ED2. In this case, the subtraction image data set SBD1 of the first result data set ED1 may be determined as the difference between a first image data set BD1 and in each case a maximum opacity image MOP. Analogously to this, the subtraction image data set SBD2 of the second result data set ED2 may be determined as the difference between a first image data set BD1 and in each case a maximum opacity image MOP. In FIG. 7, the respective differences are represented as bright, thus, for example, a reperfused brain area of the object under examination.

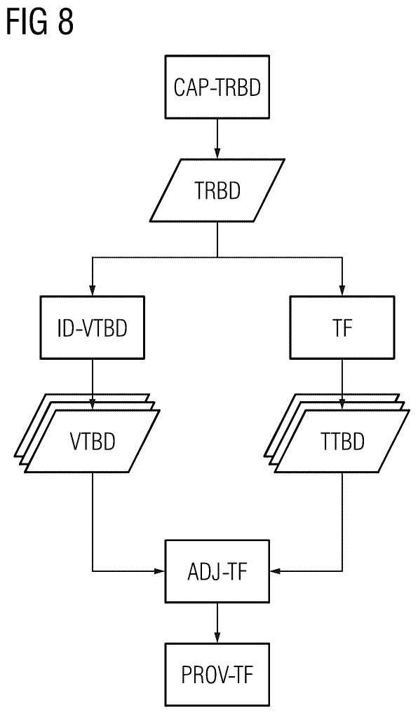

FIG. 8 shows a schematic representation of an advantageous embodiment of a proposed method for providing a trained function PROV-TF. In a first act, a medical training image data set TRBD may be captured CAP-TRBD, which maps a flow of contrast agent in an object under examination in a time-resolved manner. In a further act, multiple comparison partial image data sets VTBD may be identified ID-VTBD in the training image data set TRBD. The comparison partial image data sets VTBD may in each case map one of multiple physiological subphases. Further, the comparison partial image data sets VTBD may be identified ID-VTBD on the basis of their respective acquisition times and/or on the basis of differences between the respectively mapped flow of contrast agent and/or by annotation in the training image data set TRBD. In a further act, multiple training image data sets TTBD may be identified by applying the trained function TF to input data. In this case, the input data of the trained function TF may be based on the training image data set TRBD. Further, the training image data sets TTBD may be provided as output data of the trained function TF. In a further act, at least one parameter of the trained function TF may be adjusted ADJ-TF on the basis of a comparison of the training image data sets TTBD with the comparison partial image data sets VTBD. After this, the trained function TF may be provided PROV-TF.

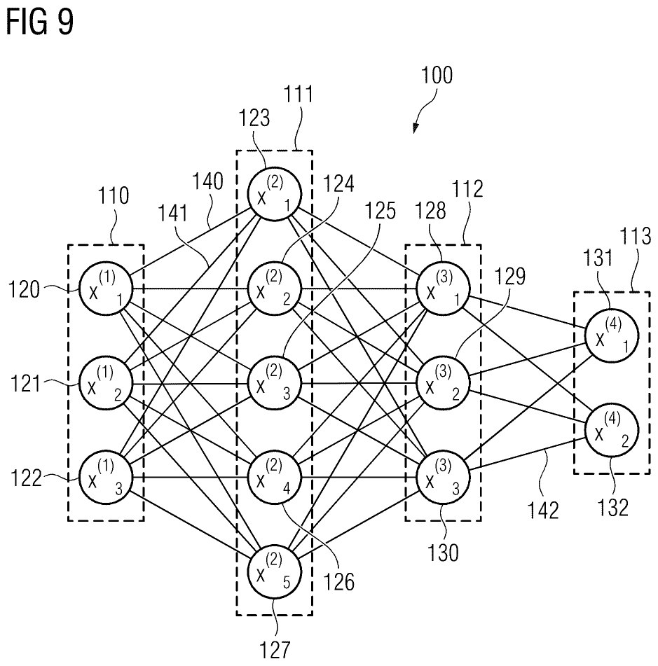

FIG. 9 shows a schematic representation of an artificial neural net 100, as may be employed in a method in accordance with FIG. 5. The neural net may also be designated as an artificial neural net, artificial neural network, or neural network.

The neural net 100 includes nodes 120, . . . , 129 and edges 140, 141, wherein each edge 140, 141 is a directed connection from a first node 120, . . . ,129 to a second node 120, . . . , 129. The first node 120, . . . ,129 and the second node 120, . . . ,129 may be different nodes. Alternatively, it is also possible for the first node 120, . . . ,129 and the second node 120, . . . , 129 to be identical. An edge 140, 141 from a first node 120, . . . ,129 to a second node 120, . . . ,129 may also be designated as an inward edge for the second node and as an outward edge for the first node 120, . . . , 129.

The neural net 100 responds to input values x⁽¹⁾₁, x⁽¹⁾₂, x⁽¹⁾₃ for a plurality of input nodes 120, 121, 122 of the input layer 110. The input values x⁽¹⁾₁, x⁽¹⁾₂, x⁽¹⁾₃ are used to generate one or a plurality of outputs x⁽³⁾₁, x⁽³⁾₂, The node 120 is for example connected to the node 123 via an edge 140. The node 121 is for example connected to the node 123 via the edge 141.

The neural net 100 learns in this exemplary embodiment, in that it adjusts the weighting factors w_{i,j} (weights) of the individual nodes on the basis of training data. Possible input values x⁽¹⁾₁, x⁽¹⁾₂, x⁽¹⁾₃ of the input nodes 120,121,122 may be the training image data sets TRBD.

The neural net 100 weights the input values of the input layer 110 on the basis of the learning process. The output values of the output layer 112 of the neural net 100 may correspond to a classification of the X-ray acquisition. The output may take place via one individual or a plurality of output nodes x⁽³⁾₁, x⁽³⁾₂ in the output layer 112.

The artificial neural net 100 may include a hidden layer 111, which includes a plurality of nodes x⁽²⁾₁, x⁽²⁾₂, x⁽²⁾₃. Multiple hidden layers may be provided, wherein a hidden layer uses output values of another hidden layer as input values. The nodes of a hidden layer 111 perform mathematical operations. An output value of a node x⁽²⁾₁, x⁽²⁾₂, x⁽²⁾₃ in this case corresponds to a non-linear function f of its input values x⁽¹⁾₁, x⁽¹⁾₂, x⁽¹⁾₃ and the weighting factors w_{i,j}. After the receipt of input values x⁽¹⁾₁, x⁽¹⁾₂, x⁽¹⁾₃ a node x⁽²⁾₁, x⁽²⁾₂, x⁽²⁾₃ carries out a summation of a multiplication, weighted with the weighting factors W_{i,j}, of each input value x⁽¹⁾₁, x⁽¹⁾₂, x⁽¹⁾₃, as determined by the following function:

$${x}_{j}^{( n + 1 )} = f ( {\sum }_{i} {x}_{i}^{( n )} \cdot {w}_{i , j}^{( n )} ) .$$

The weighting factor w_{i,j} may be a real number, in particular may lie in the interval of [−1;1] or [0;1]. The weighting factor w_{i,j}^{(m,n)} designates the weight of the edge between the i-th node of an m-th layer 110,11,112 and a j-th node of the n-th layer 110,111,112. The weighting factor w_{i,j}^{(m,n)} an abbreviation for the weighting factor w_{i,j}^{(n,n+1)}.

In particular, an output value of a node x⁽²⁾₁ x⁽²⁾₂ x⁽²⁾₃ is formed as a function f of a node activation, (e.g., a sigmoidal function or a linear ramp function). The output values x⁽²⁾₁, x⁽²⁾₂, x⁽²⁾₃ are transferred to the output node or nodes 128,129. Once again, a summation of a weighted multiplication of each output value x⁽²⁾₁, x⁽²⁾₂ x⁽²⁾₃ is calculated as a function of the node activation f and thus the output values x⁽³⁾₁, x⁽³⁾₂.

The neural net 100 shown here is a feedforward neural net, in which all nodes 111 process the output values of a previous layer in the form of its weighted total as input values. Other types of neural net may of course also be employed in accordance with the disclosure, for example feedback nets, in which an input value of a node may simultaneously also be its output value.

The neural net 100 is trained to recognize patterns by a supervised learning method. A known procedure is backpropagation, which may be applied for all embodiments disclosed herein. During the training, the neural net 100 is applied to input training data or values and generates corresponding, previously known output training data or values. Mean square errors (MSE) between calculated and expected output values are calculated iteratively and individual weighting factors are adjusted until the deviation between calculated and expected output values lies below a predetermined threshold.

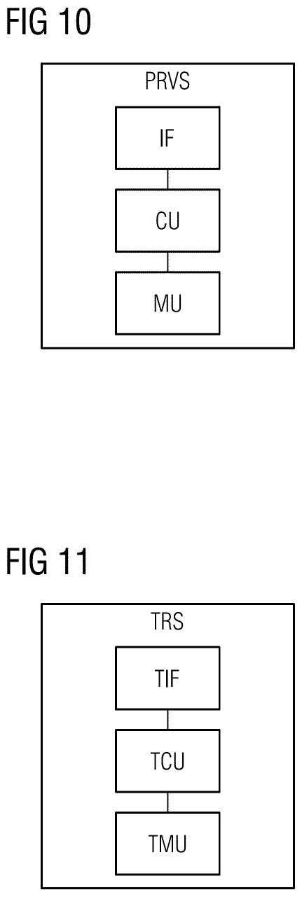

FIG. 10 shows a schematic representation of a provision unit PRVS. In this case, the provision unit PRVS may include a computing unit CU, a memory unit MU and/or an interface IF. The provision unit PRVS may be configured to perform a proposed method for providing a result data set PROV-ED and/or for providing a comparison data set PROV-CD, in that the interface IF, the computing unit CU, and/or the memory unit MU are configured to perform the corresponding method acts.

FIG. 11 shows a schematic representation of a training unit TRS. In this case, the training unit TRS may advantageously include a training interface TIF, a training memory unit TMU, and/or a training computing unit TCU. The training unit TRS may be configured to perform a method for the provision PROV-TF of a trained function TF, in that the training interface TIF, the training memory unit TMU, and/or the training computing unit TCU are configured to perform the corresponding method acts.

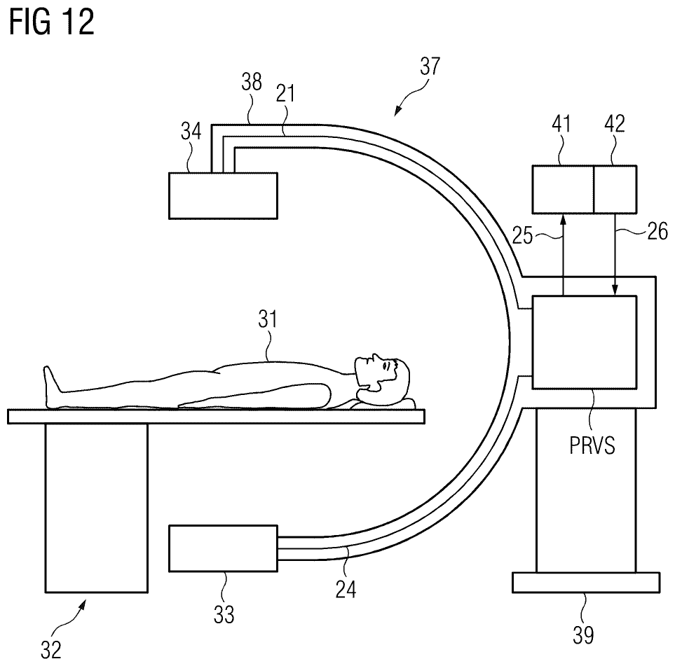

FIG. 12 shows by way of example of a medical imaging device a schematic representation of a medical C-arm X-ray device 37, including a proposed provision unit PRVS. The medical C-arm X-ray device 37 may advantageously have a detector 34, (e.g., an X-ray detector), and a source 33, (e.g., an X-ray source), which are arranged in a defined arrangement on a C-arm 38. The C-arm 38 of the C-arm X-ray device 37 may be mounted so as to move about one or more axes. To acquire the first and second image data set BD1 and BD2 of the object under examination 31, positioned on a patient positioning device 32, the provision unit PRVS may send a signal 24 to the X-ray source 33. The X-ray source 33 may then emit an X-ray beam. When the X-ray beam, following an interaction with the object under examination 31, hits a surface of the detector 34, the detector 34 may send a signal 21 to the provision unit PRVS. The provision unit PRVS can, on the basis of the signal 21, capture CAP-BD1 and CAP-BD2 the first and second image data set BD1 and BD2.

The system may further have an input unit 42, (e.g., a keyboard), and a display unit 41, (e.g., a monitor and/or a display and/or a projector). The input unit 42 may be integrated into the display unit 41, for example, in the case of a capacitive and/or resistive input display. The input unit 42 may advantageously be configured to capture a user input. For this, the input unit 42 may send a signal 26 to the provision unit PRVS. The provision unit PRVS may be configured to be controlled as a function of the user input, in particular of the signal 26, in particular for the performance of a method for providing a result data set PROV-ED and/or for providing a comparison data set PROV-CD. In particular, the times of the commencement and/or end of the physiological subphases, which for example are predetermined automatically, may be corrected manually on the basis of the user input, in order to improve the accuracy of the subdivision of the second image data set BD2, in particular the DSA series, into the physiological subphases.

The display unit 41 may advantageously be configured to display a graphical representation of the result data set ED and/or of the comparison data set CD. For this, the provision unit PRVS may send a signal 25 to the display unit 41.

The schematic representations contained in the figures described do not depict any scale or proportions.

In conclusion, it is again noted that the methods described, and devices represented in detail above relate solely to exemplary embodiments that may be modified by the person skilled in the art in a variety of ways, without departing from the scope of the disclosure. Further, the use of the indefinite article “a” or “an” does not rule out that the features in question may also be present multiple times. Likewise the terms “unit” and “element” do not rule out that the components in question multiple interacting subcomponents that, if appropriate, may also be distributed spatially.

The expression “on the basis of” may be understood in the context of the present application in particular in the meaning of the expression “using.” In particular, a wording in accordance with which a first feature is generated (alternatively: determined, ascertained, etc.) on the basis of a second feature, does not rule out that the first feature may be generated (alternatively: determined, ascertained, etc.) on the basis of a third feature.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for providing a result data set, the method comprising:
capturing a first medical image data set that maps an object under examination within a first temporal phase;
capturing a second medical image data set that maps a flow of contrast agent in the object under examination within a second temporal phase in a time-resolved manner;
identifying multiple partial image data sets in the second medical image data set, wherein each partial image data set of the multiple partial image data sets maps one of multiple physiological subphases within the second temporal phase; and
providing the result data set comprising multiple subtraction image data sets,
wherein each subtraction image data set of the multiple subtraction image data sets is determined based on the first medical image data set and a respective partial image data set of the multiple partial image data sets in the second medical image data set.

2. The method of claim 1, wherein each subtraction image data set of the multiple subtraction image data sets is determined as a difference between the first medical image data set and a respective partial image data set of the multiple partial image data sets.

3. The method of claim 1, wherein the providing of the result data set comprises determining a maximum opacity image for each partial image data set of the multiple partial image data sets, which image point by image point has a maximum opacity within a respective physiological subphase along a temporal dimension, and
wherein each subtraction image data set of the multiple subtraction image data sets is determined as a difference between the first medical image data set and a respective maximum opacity image of the maximum opacity images.

4. The method of claim 1, wherein the physiological subphases comprise an arterial phase, a parenchymal phase, a venous phase, or a combination thereof.

5. The method of claim 1, wherein the identifying of the multiple partial image data sets comprises applying a trained function to input data,
wherein the input data is based on the second medical image data set,
wherein at least one parameter of the trained function is adjusted based on a comparison of training partial image data sets with comparison partial image data sets, and
wherein the multiple partial image data sets are provided as output data of the trained function.

6. The method of claim 1, wherein the providing of the result data set comprises displaying a graphical representation of the result data set by a display unit.

7. The method of claim 6, wherein the graphical representation of the result data set comprises a color-coded representation, a superimposed representation, a coordinated representation, a sequential representation, or a combination thereof of the subtraction image data sets.

8. The method of claim 1, wherein the providing of the result data set comprises a registration of each partial image data set to be subtracted and of the first medical image data set.

9. The method of claim 1, wherein the multiple partial image data sets are identified in the second medical image data set based on respective acquisition times of the multiple partial image data sets.

10. The method of claim 1, wherein the multiple partial image data sets are identified based on differences between a flow of the contrast agent mapped in each case.

11. A method for providing a comparison data set, the method comprising:
providing a first result data set by, at a first time: capturing a first medical image data set that maps an object under examination within a first temporal phase; capturing a second medical image data set that maps a flow of contrast agent in the object under examination within a second temporal phase in a time-resolved manner; and identifying multiple partial image data sets in the second medical image data set, wherein each partial image data set of the multiple partial image data sets maps one of multiple physiological subphases within the second temporal phase, wherein the first result data set comprises multiple subtraction image data sets, and wherein each subtraction image data set of the multiple subtraction image data sets is determined based on the first medical image data set and a respective partial image data set of the multiple partial image data sets in the second medical image data set;
providing a second result data set by, at a second time: capturing an additional first medical image data set that maps the object under examination within the first temporal phase; capturing an additional second medical image data set that maps the flow of the contrast agent in the object under examination within the second temporal phase in the time-resolved manner; and identifying additional multiple partial image data sets in the additional second medical image data set, wherein each partial image data set of the additional multiple partial image data sets maps one physiological subphase of multiple physiological subphases within the second temporal phase, wherein the second result data set comprises additional multiple subtraction image data sets, wherein each subtraction image data set of the additional multiple subtraction image data sets is determined based on the additional first medical image data set and a respective partial image data set of the additional multiple partial image data sets in the additional second medical image data set, and wherein a change in the object under examination has taken place between the first time and the second time; and
providing the comparison data set comprising in each case a difference between a subtraction image data set of the first result data set and of the second result data set that map a same physiological subphase.

12. A computer-implemented method for providing a trained function, the method comprising:
capturing a medical training image data set that maps a flow of contrast agent in an object under examination in a time-resolved manner;
identifying multiple comparison partial image data sets in the medical training image data set, wherein each comparison partial image data set maps one of multiple physiological subphases, wherein the multiple comparison partial image data sets are identified based on: respective acquisition times of each respective comparison partial image data set, based on differences between the respectively mapped flow of the contrast agent, by annotation in the medical training image data set, or combinations thereof,
identifying multiple training image data sets by applying the trained function to input data, wherein the input data is based on the training image data set, and wherein training partial image data sets are provided as output data of the trained function;
adjusting at least one parameter of the trained function based on a comparison of the multiple training partial image data sets with the comparison partial image data sets; and
providing the trained function.

13. A medical imaging device comprising:
a provision unit,
wherein the medical imaging device is configured to:
capture a first medical image data set that maps an object under examination within a first temporal phase; and
capture a second medical image data set that maps a flow of contrast agent in the object under examination within a second temporal phase in a time-resolved manner, and
wherein the provision unit of the medical imaging device is configured to:
identify multiple partial image data sets in the second medical image data set, wherein each partial image data set of the multiple partial image data sets maps one of multiple physiological subphases within the second temporal phase; and
provide a result data set comprising multiple subtraction image data,
wherein each subtraction image data set of the multiple subtraction image data sets is determined based on the first medical image data set and a respective partial image data set of the multiple partial image data sets in the second medical image data set.
